(19) United States
(12) Patent Application Publication
(10) Pub. No.: US 2026/0183537 A1
(43) Pub. Date: Jul. 2, 2026

(54) ARTIFICIAL RETINA DEVICE

(71) Applicant: THE TRUSTEES OF INDIANA UNIVERSITY, Bloomington, IN (US)

(72) Inventors: Afshin IZADIAN, Carmel, IN (US); Amir Reza HAJRASOULIHA, Carmel, IN (US)

(21) Appl. No.: 19/129,939

(22) PCT Filed: Nov. 30, 2023
(86) PCT No.: PCT/US2023/081813

§ 371 Date: May 14, 2025

Related U.S. Application Data
(60) Provisional application No. 63/385,698, filed on Dec. 1, 2022.

(51) Int. Cl. A61N 1/05; A61N 1/36; A61N 5/06

(52) U.S. Cl. CPC A61N 1/0543; A61N 1/36125; A61N 1/36128; A61N 5/0622; A61N 2005/0663

(57) ABSTRACT

The present disclosure is directed to systems and methods for treating retinal dystrophy and retinal degenerations. The disclosure describes retina device comprising a first component including an energy conversion and an interface layer comprising gold nanoparticles and a second component including a light-shutter valve (LSV) configured to operate as a neurostimulator.

### CROSS REFERENCE TO RELATED APPLICATIONS

This application claims priority to U.S. Provisional Patent Application No. 63/385,698, filed on Dec. 1, 2022, the disclosure of which is expressly incorporated herein.

### TECHNICAL FIELD

The present disclosure relates generally to devices and methods for treating retinal dystrophy and/or retinal degenerations. More specifically, the present disclosure relates to systems and methods for mimicking photoreceptor operations, artificially stimulating optical nerves, providing a non-toxic interface, and/or improving the longevity of retinal ganglion cells and improving vision.

### BACKGROUND

Photoreceptor loss resulting is low vision is common in retinal dystrophy and/or retinal degenerations. Additionally, hereditary dystrophy affecting the retina in children and adults is the leading cause of blindness.

Despite the loss of photoreceptors, an appreciable number of retinal ganglion cells (RGCs) remain healthy. Thus, electrical stimulation can be utilized as a strategy to restore vision. Nanotechnology methods can be employed to treat retinal diseases. Nanoparticles are designed to convert visible light to high enough voltage or heat to excite the optical nerves. However, these nanoparticles must stay in a constant contact to the retina and be interfaced with the retinal ganglion cells at a frequency to correctly excite the nerves and prevent their fatigue and death.

Current technology for RGC stimulation relies on the existence of an external camera to process the pictures and a limited number of implanted electrodes in the eye to stimulate the nerves. This requires an external camera, image processing units, external energy sources, and provides limited resolution while generating a considerable amount of heat in the eye.

The present disclosure is directed to devices and methods of using the devices that can mimic photoreceptor operations without the need for external power or camera. They are comprised of two or more part components. The first part can adaptively mimic photoreceptor operations, does not a need camera interface with the in-eye unit, does not need a power source in the eye, and can restore high-resolution vision. The second component can provide a non-toxic interface while improving the longevity of RGCs.

### SUMMARY

Systems and methods for treating retinal dystrophy and/or retinal degeneration are described. Importantly, a color-coded approach to restoring high-resolution colored vision and improving the longevity of RGCs using a non-toxic interface is described.

In a first aspect, the present disclosure is directed to an artificial retina device includes a first and a second component. The first component is an energy conversion and interface layer comprising gold nanoparticles and a composite dielectric layer, and the second component is a light shutter valve. The first component is implanted in a patient or user, and the second component may be separate from the first component.

In some embodiments, wherein the first component further comprises a composite dielectric layer. In some embodiments, the dielectric layer comprises one or more of polyvinylidene fluoride (PVDF), polyvinylpyrrodidone (PVP), polyethylene glycol (PEG), and BaTiO₂.

In some embodiments, the second component is configured to can be controlled to open and close on demand and stimulate retinal ganglion cells (RGCs) on demand. In some embodiments, the first component and the second component do not require an external power source. In some embodiments, the device is configured to create a colored picture on a retina of the patient.

In some embodiments, the first component is injectable. In some embodiments, the LSV includes a layer of TiO, a layer of polyvinyl fluoride (PVF), and a liquid crystal film sandwiched between two transparent substrates. In some embodiments, the second component can be operated manually or automatically. In some embodiments, the size of the gold nanoparticles ranges from about from 5 nm to about 50 nm.

In a second aspect, the present disclosure is directed to a method of using a retina device comprising determining an intensity of incident light on a retina, and operating a light shutter valve at a frequency configured to align with the intensity of the incident light. An increase in frequency increases a nerve excitation pulse on the retina.

In some embodiments, the method further comprises aligning a color of the incident light with the operation of the light shutter. In some embodiments, the method further comprises utilizing the gold nanoparticles for artificially replacing natural photoreceptors and stimulating the retinal ganglion cells (RGCs) in a patient. In some embodiments, the method further comprises stimulating the RGCs in a regulated manner.

In a second aspect, the present disclosure is directed to a method of implementing a retina device comprising receiving a controlled hue of light by utilizing an conversion and interface layer, generating a voltage with the intensity and duration enough to excite retinal ganglion cells (RGCs), and correcting vision in a user by utilizing an energy conversion and interface layer and a light shutter valve (LSV).

In some embodiments, the method further comprises generating negligible heat. In some embodiments, the method further comprises implementing an individually operated micro light valve that mimics the operation of a photoreceptor frequency in the LSV for correcting vision in the user. In some embodiments, the method further comprises operating the LSV for controlling the hue of light. In some embodiments, the method further comprises adjusting stimulation of the energy conversion and interface layer based on the pathology and severity of the disease in the eye. In some embodiments, the method further comprises stimulating the RGCs in a regulated manner.

### BRIEF DESCRIPTION OF DRAWINGS

The following description accompanies the drawing(s), all given by way of non-limiting examples that may be useful to understand how the described method and composition may be embodied.

FIG. 1A is an illustration of one embodiment of an artificial retina device comprising a light shutter valve (LSV) and a gold-nanoparticle neurosensory electro-stimulation (GNES) coupled together;

FIG. 1B is an illustration of one embodiment of the artificial retina device comprising the LSV and the GNES configured to be independent from each other;

FIG. 2A is an illustration of the embodiment of the artificial retina device illustrated in FIG. 1B, where the LSV allows the passage of light;

FIG. 2B is an illustration of the embodiment of the artificial retina device illustrated in FIG. 1B, where the LSV does not allow the passage of light;

FIG. 3 is a graph illustrating the base of the GNES transformation of light to electricity;

FIG. 4 is an illustration of dynamic light scattering of the GNES based on citrate concentration used in nanoparticle synthesis;

FIG. 5 is an illustration of coated gold nanoparticle injected into the blind mice recover the electrical activity of retina; and

FIG. 6 shows data identifying the need for a shutter or the LSV for nerve network activity.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. Accordingly, aspects and features of every embodiment may not be described with respect to each embodiment, but those aspects and features are applicable to the various embodiments unless statements or understandings are to the contrary.

As used herein, the term “patient” or “user” refers to any subject including mammals and humans. The patient may have a disease or suspected of having a disease and as such is being treated with a drug. In some instances, the patient is a mammal, such as a human, a premature neonate, neonate, infant, juvenile, adolescent, or adult thereof. In some instances, the term “patient,” as used herein, refers to a human (e.g., a man, a woman, or a child). In some instances, the term “patient,” as used herein, refers to laboratory animal of an animal model study. The patient or subject may be of any age, sex, or combination thereof.

The term “treating” refers to administering a therapy in an amount, manner, or mode effective (e.g., a therapeutic effect) to improve a condition, symptom, disorder, or parameter associated with a disorder, or a likelihood thereof.

The terms “essentially” or “substantially” as used herein mean to a great or significant extent, but not completely.

The term “about” as used herein refers to any values, including both integers and fractional components that are within a variation of up to ±10% of the value modified by the term “about.”

The present disclosure is directed to a device or implant and methods of using the device that can selectively convert a visible light spectrum to stimulate retinal ganglion cells. The device can substitute loss of function in photoreceptors, and directly activate retinal ganglion cells (RGCs). The device can be implanted through injection into the vitreous cavity or vitrectomy, i.e., eye surgery over a diseased retina at the back of an eye of a patient or user. The device can be attached to the ganglion cells of the retina. The implantation can be verified with intraoperative imaging of the layers of the retina during the surgery and/or after surgery. The device may be biocompatible and may need no external energy. The device may use the plasmonic effect of nanoparticles to convert specific wavelengths of light to an action potential. The action potential may be high enough to activate the optical nerves. The device may partially restore the vision in blind patients.

In one embodiment, dielectric physics properties of nanoscale material can be used to generate action potential at a specific projected light's wavelength. Additionally, or alternatively, a surface interface of the nanoparticles and RGCs can be improved for a maximum action potential response and/or a specific color-coded response. The device can allow the activation of RGCs sustainably on a large area of contact allowing high resolution and increasing RGCs cell survival.

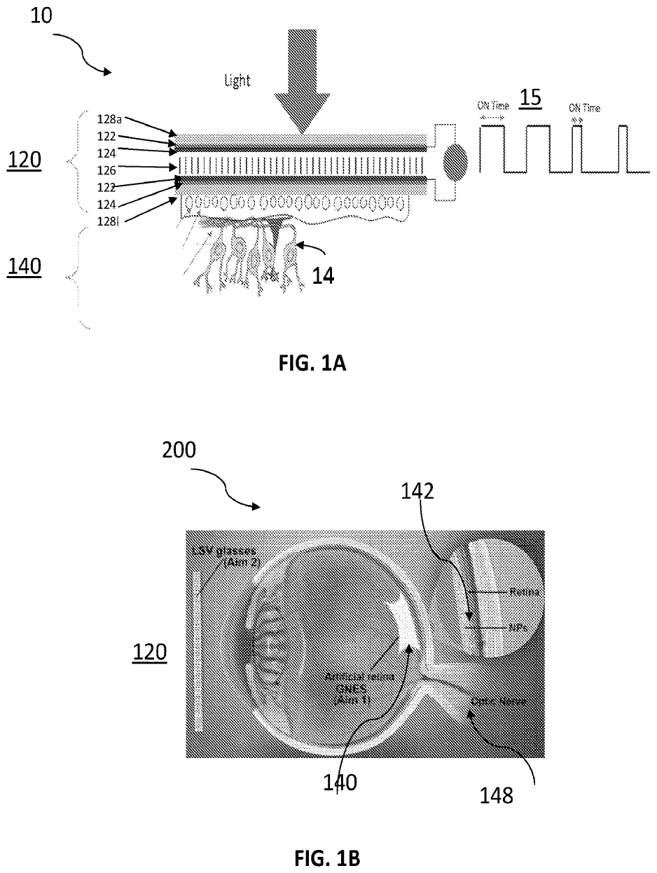

As shown in FIGS. 1A and 1B, an artificial retina device 100, 200 can artificially replace photoreceptors and excite RGCs 148 in a regulated manner. The device 100, 200 can include a first component or portion, an energy conversion and interface layer or autonomous gold-nanoparticle neurosensory electro-stimulation (GNES) 140 and a second component or portion, a light valve shutter or light-shutter valve (LSV) 120. Utilization, implementation, and/or control of the LSV 120 enables the device 100, 200 to deliver a high-resolution image and cause lasting neurostimulation.

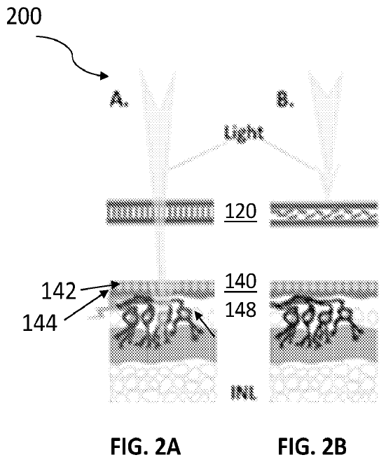

As shown in FIG. 1B, the LSV 120 can be configured, designed, utilized, operated, and/or implemented as a stand-alone neurostimulator to stimulate RGCs 148. The presence of the LSV 120 can improve the excitatory capability of the device 200 due to the frequency and duration of stimulation. The light intensity of a particular point in a picture can be converted into the duration and frequency of the nerve stimulation. The LSV 120 can be controlled to open and close on demand, and can thus stimulate RGCs 148 on demand. The LSV 120 can be operated manually or automatically. The LSV 120 can consist of one or more individually operated micro light valves that mimic the operation of the photoreceptors frequency. Since photoreceptors reset their operation even under constant light exposure during their natural operation, utilization, and/or implementation of the LSV 120 can increase RGC 148 survival. In the exemplary embodiment shown in FIGS. 1B, 2A, and 2B, the LSV 120 is an external component and separate from the energy conversion and interface layer or GNES 140. In some embodiments, the LSV 120 may be wearable (e.g., contact lens, glasses, etc.). In some embodiments, the LSV 120 can be utilized and/or implemented with other energy conversion and interface layers 140 not described herein. In yet other embodiments, as shown in FIG. 1A, one or more components of the energy conversion and interface layer or GNES 140 (e.g., gold nanoparticles 142) can be attached or coupled to the LSV 120.

As shown in FIGS. 1A, 2A, and 2B, the LSV 120 can include layers of TiO 122, polyvinyl fluoride (PVF) 124, and liquid crystal film 126 sandwiched between two transparent substrates 128a, 128b. When voltage is applied to the liquid crystal film 126, it may turn transparent, else the liquid crystal film 126 may be opaque. The LSV 120 can be voltage-activated and can operate at frequencies close to the natural nerve excitation rate of about 24 Hz to about 30 Hz, including any frequency or range comprised therein. Alternatively, the liquid crystal film 126 may be transparent in the absence of any voltage application and may become opaque when voltage is applied. By tuning the light using the LSV 120, the exposure time and heat generation time at the RGCs 148 may be limited. Such tuned exposure may result in long lasting high resolution images and cause minimal toxicity to the RGCs 148. In other embodiments, the LSV 120 may comprise other electrically operated light sensitive materials. The size of the LSV 120 is such that it covers the pupil to regulate the light entering the eye and exposure to the retina.

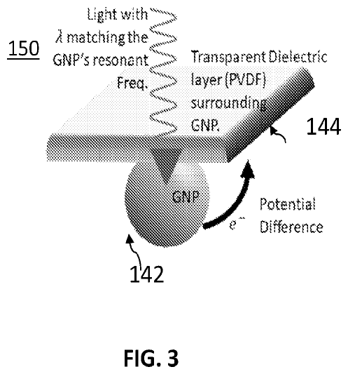

The energy conversion and interface layer 140 can be an implantable component that can be placed inside the eye to restore vision in the user. The energy conversion and interface layer 140 can comprise gold nanoparticles 142 to proportionally generate stimulation under visible light and regulate the stimulation of RGCs 148 by the LSV 120. In other embodiments, the energy conversion and interface layer 140 can comprise nanoparticles that are surrounded or coated by dielectric materials of proper type and size. The regulation and/or excitation of RGCs 148 activity by the device 100, 200 is critical for image formation. Each gold nanoparticle 142 in the energy conversion and interface layer or GNES 140 can be of different size or of the same size. In one embodiment, monodispersed (same size) gold nanoparticles 142 can be tuned for a specific light color which can generate enough excitation to reach action potential of the RGCs 148. As shown in FIG. 3, when the resonant frequency of the gold nanoparticles 142 and the wavelength of the light match or align, a plasmo-electric effect 160 occurs.

The size of the gold nanoparticles 142 can be selected such that the gold nanoparticles 142 resonate at a specific light wavelength. In some embodiments, the size of the gold nanoparticles 142 may be about 20 nm, and the gold nanoparticles 142 may resonate at a wavelength close to green light. Alternatively, the gold nanoparticles 142 may be sized to function at different wavelengths, corresponding to red light, blue light, or a combination. The size of the gold nanoparticles 142 may be based on the wavelength of the corresponding light and may range from about from 5 nm to about 50 nm, from about 50 nm to about 100 nm, from about 100 nm to about 200 nm, including any wavelength or range of wavelength comprised therein.

The gold nanoparticles 142 may be arranged, embedded, attached, covered, or coated on or with a dielectric layer 144. The arrangement or placement of the gold nanoparticles 142 on the dielectric layer 144 may be random or according to a pattern. If the gold nanoparticles 142 are embedded within or placed on the dielectric layer 144, then the gold nanoparticles 142 can convert any absorbed light energy into a cloud of electrons. The dielectric layer 144 generates voltage potential and also interface with the optic nerve 146. The heat generated from plasmonic effect of the gold nanoparticles 142 can be transferred to an internal liquid in the eye as it has a transparent heat conductor layer. Thus, the dielectric layer 144 can also function as a heat shield to protect the eye from the generated heat. The dielectric layer 144 may comprise any or combination of polyvinylidene fluoride (PVDF), polyvinylpyrrodidone (PVP), polyethylene glycol (PEG), BaTiO₂ or any other dielectric layer.

In some embodiments, the dielectric layer 144 may be an activated dielectric layer. An activated dielectric layer provides a faster reaction time and significantly increases the light to voltage conversion efficiency. In some embodiments, the gold nanoparticles 142 may be sandwiched between two dielectric layers 144. In some embodiments, the energy conversion and interface layer or GNES 140 may comprise multiple layers of the gold nanoparticles 142 and/or the dielectric layers 144. In other embodiments, the energy conversion and interface layer or GNES 140 may comprise multiple sandwiched layers of the gold nanoparticles 142 within two or more dielectric layers 144. Using multiple layers of the gold nanoparticles 142 and/or the dielectric layers 144 can increase the potential of the energy conversion and interface layer or GNES 140. In some embodiments, the energy conversion and interface layer or GNES 140 can be flexible, biocompatible, and or reversible. In some embodiments, the energy conversion and interface layer or GNES 140 can comprise a retinal glue. In other embodiments, magnetic propagation can be utilized and/or implemented to situate the energy conversion and interface layer or GNES 140 on the retina.

The present disclosure describes a method of utilizing the gold nanoparticles 142 for artificially replacing natural photoreceptors in the eye and exciting or stimulating the RGCs 148 in a regulated manner. The method can include converting light to a high enough voltage in the range of millivolts to excite and open the calcium channels in the eye. The method can include utilizing one or more layers of the dielectric layer 144 and gold nanoparticles 142 as the energy conversion and interface layer or GNES 140 and the LSV 120 to regulate stimulation of the RGCs 148.

The LSV device 100, 200 may require to be controlled and powered. The power can be fed from any energy storage unit (e.g., bio-batteries, batteries, etc.). Alternatively, or additionally, the device 100, 200 may power itself. The device 100, 200 can use a solar cell or a conductive layer with differently sized gold nanoparticles 142. As shown in FIGS. 1A, 2A, and 2B, the LSV 120 can be utilized and/or operated to send pulses 150 of light to the gold nanoparticles 142. The pulses 150 can be generated at a specific frequency, and the duration of exposure of the gold nanoparticles 142 to the pulses 150 can be controlled by an ON time of the pulse 150. The longer the exposure, the more is the energy that is received by the gold nanoparticles 142 in the energy conversion and interface layer or GNES 140. When the pulse 150 is ON, the liquid crystal film 126 can become transparent, pass light to the gold nanoparticles 142, and activate the optic nerve 146. Once the pulse 150 is OFF, the liquid crystal film 126 can become opaque, block light, and reset the activated optic nerve 146. The duty cycle of the pulse 150 can be controlled to adjust for intensity of light. The duty cycle of the pulse 150 can be controlled by a closed loop system or by an external command from a person or a controller. The duty cycle of the pulse 150 can control the amount of heat generated by the device 100, 200. FIG. 2A illustrates an open LSV 120, allowing passage of light and excitation of the RGCs 148, while FIG. 2B illustrates a close LSV 120, and no excitation of the RGCs 148.

The method can include determining the size of the gold nanoparticles 142 based on stimulating wavelength. The method can include depositing the gold nanoparticles 142 at a distance optimized for enhancing image resolution and at a distance that is based on the probability of exciting nerves. Alternatively, or additionally, the method can include utilizing an activated dielectric layer 144 to create an initial excitation. The method can include the minimal initial excitation reaching the action potential of the optic nerve 146 in activation time and utilizing a permanent electrostatic field built in the dielectric layer 144.

The present disclosure describes a method of utilizing the energy conversion and interface layer or GNES 140 and the LSV 120 for correcting vision in a user. The method can include the energy conversion and interface layer or GNES 140 receiving a controlled hue of light and generating a voltage with the intensity and duration enough to excite the RGCs 148 with negligible heat generation effect. The method can include utilizing or implementing the energy conversion and interface layer or GNES 140 and one or more individually operated micro light valves that mimic the operation of the photoreceptors frequency in the LSV 120 for correcting vision in the user.

The method can include the LSV 120 controlling the hue of light. The method can include the LSV 120 adjusting stimulation of the energy conversion and interface layer or GNES 120 based on the pathology and severity of the disease in the eye. The method can include utilizing the LSV 120 to reduce heat generation at the RGCs 148.

### EXAMPLES AND METHODOLOGY

### Example 1: Evaluation of Size of the Gold Nanoparticles

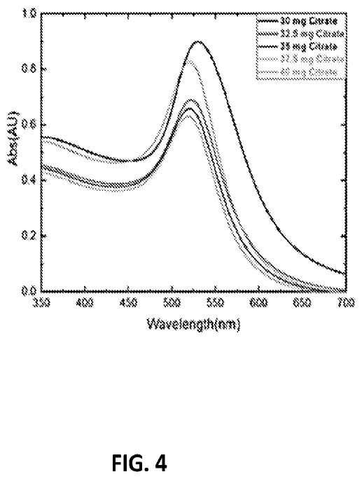

Following the synthesis of the nanoparticles 142, their size was evaluated. As shown in FIG. 4, monodispersity as well as uniformity was observed with both electron microscopy (TEM) and Dynamic Light Scattering (DLS) methods. As evident, the citrate timing and concentration have a great impact on the monodispersity of the sample. It was noted that when the amount of citrate in the solution was 35 mg, the size of gold nanoparticles 142 changed randomly over time, resulting in particles ranging from less than 1 nm to about 30 nm for different time points. The most uniform result corresponds to the sample taken after 10 minutes at 37.5 mg of citrate with 97.7 percent at 21 nm size. The maximum absorption of the gold nanoparticles 142 was in the green range, between 525 to 535 nm.

### Example 2: Retinal Activity in Blind Mice

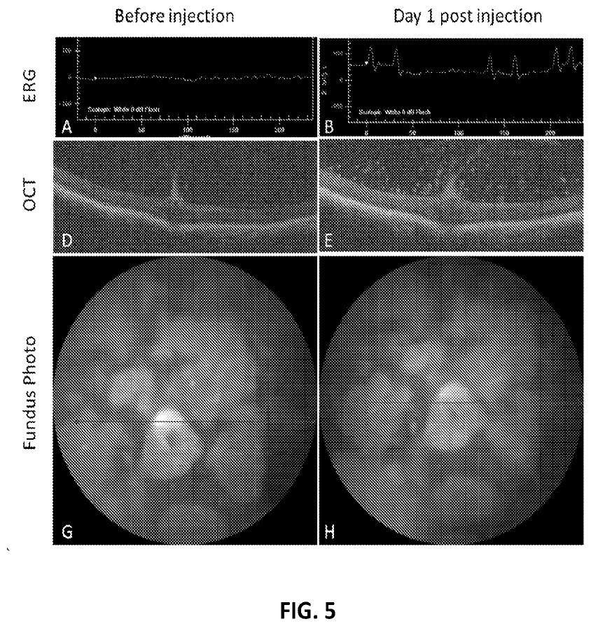

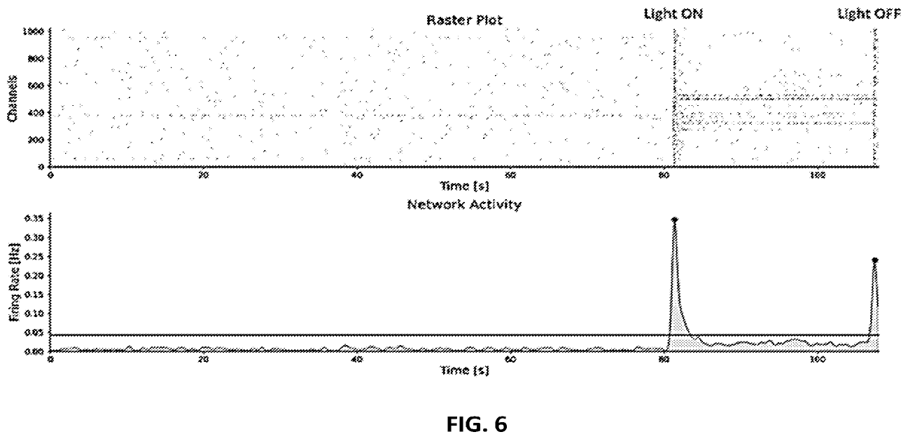

Gold nanoparticles 142 synthesized as described above were injected into the retina of blind mice (Pde6b) as shown in FIG. 5. Nerve activity was recorded by MEA on the mice's extracted retina both before and after injection of the gold nanoparticles. The ERG shows higher nerve activity of the blind mice retina one day after injection compared to prior to injection. The nerve light source was turned on and off before and after recording the nerve activity. FIG. 5 also shows the presence of nanoparticles inside the vitreous cavity after injection. As illustrated in FIG. 6, the use of a shutter or the LSV 120 is necessary for nerve network activity.

The figures provided herein are not necessarily to scale, although a person skilled in the art will recognize instances where the figures are to scale and/or what a typical size is when the drawings are not to scale. While in some embodiments movement of one component is described with respect to another, a person skilled in the art will recognize that other movements are possible. Additionally, a number of terms may be used throughout the disclosure interchangeably but will be understood by a person skilled in the art. Further, to the extent features, sides, or steps are described as being “first” or “second,” such numerical ordering is generally arbitrary, and thus such numbering can be interchangeable. Still further, in the present disclosure, like-numbered components of various embodiments generally have similar features when those components are of a similar nature and/or serve a similar purpose. Lastly, the present disclosure includes some illustrations and descriptions that include prototypes, bench models, or experimental design. A person skilled in the art will recognize how to rely upon the present disclosure to integrate the techniques, systems, devices, and methods provided for into a product in view of the present disclosures.

While the concepts of the present disclosure are susceptible to various modifications and alternative forms, specific exemplary embodiments of the disclosure have been shown by way of example. It should be understood, however, that there is no intent to limit the concepts of the present disclosure to the particular disclosed forms; the intention is to cover all modifications, equivalents, and alternatives falling within the spirit and scope of the invention as defined by the claims. Although this disclosure refers to specific embodiments, it will be understood by those skilled in the art that various changes in form and detail may be made without departing from the subject matter set forth in the accompanying claims.

1. An artificial retina device comprising:
a first component including an energy conversion and an interface layer comprising gold nanoparticles;
a second component including a light-shutter valve (LSV) configured to operate as a neurostimulator;
wherein the first component is implanted in a patient, and the second component is separate from the first component,
wherein the first component further comprises a composite dielectric layer selected from a group consisting of polyvinylidene fluoride (PVDF), polyvinylpyrrodidone (PVP), and BaTiO₂.

2. (canceled)

3. (canceled)

4. The retina device of claim 1, wherein the second component is configured to can be controlled to open and close on demand and stimulate retinal ganglion cells (RGCs) on demand.

5. The retina device of claim 1, wherein the first component and the second component do not require an external power source.

6. The artificial retina device of claim 1, wherein the device is configured to create a colored picture on a retina of the patient.

7. The artificial retina device of claim 1, wherein the first component is injectable.

8. The artificial retina device of claim 1, wherein the LSV includes a layer of TiO, a layer of polyvinyl fluoride (PVF), and a liquid crystal film sandwiched between two transparent substrates.

9. The retina device of claim 1, wherein the second component can be operated manually or automatically.

10. The artificial retina device of claim 1, wherein the size of the gold nanoparticles ranges from about from 5 nm to about 50 nm.

11. A method of using a retina device comprising:
determining an intensity of incident light on a retina,
operating a light shutter valve at a frequency configured to align with the intensity of the incident light,
wherein an increase in frequency increases a nerve excitation pulse on the retina.

12. The method of claim 11, further comprising aligning a color of the incident light with the operation of the light shutter.

13. The method of claim 11, further utilizing the gold nanoparticles for artificially replacing natural photoreceptors and stimulating the retinal ganglion cells (RGCs) in a patient.

14. The method of claim 13, further comprising stimulating the RGCs in a regulated manner.

15. A method of implementing a retina device comprising:
receiving a controlled hue of light by utilizing a conversion and interface layer,
generating a voltage with the intensity and duration enough to excite retinal ganglion cells (RGCs), and
correcting vision in a user by utilizing an energy conversion and interface layer and a light shutter valve (LSV).

16. The method of claim 15, further comprising generating negligible heat.

17. The method of claim 15, further comprising implementing an individually operated micro light valve that mimics the operation of a photoreceptor frequency in the LSV for correcting vision in the user.

18. The method of claim 15, further comprising operating the LSV for controlling the hue of light.

19. The method of claim 15, further comprising adjusting stimulation of the energy conversion and interface layer based on the pathology and severity of the disease in the eye.

20. The method of claim 15, further comprising stimulating the RGCs in a regulated manner.
